(19) United States
(12) Patent Application Publication
(10) Pub. No.: US 2025/0034541 A1
(43) Pub. Date: Jan. 30, 2025

(54) DRYING PROCESS OF BOTULINUM TOXIN FORMULATION

(71) Applicant: INIBIO CO., LTD., Bucheon-si Gyeonggi-do (KR)

(72) Inventors: Chung Sei KIM, Seoul (KR); Hyeona YIM, Seoul (KR); Young Je KIM, Gyeonggi-do (KR); Yeong Duk AN, Gyeonggi-do (KR)

(73) Assignee: INIBIO CO., LTD., Bucheon-si Gyeonggi-do (KR)

(21) Appl. No.: 18/709,258

(22) PCT Filed: Jul. 26, 2022
(86) PCT No.: PCT/KR2022/010991

§ 371 Date: May 10, 2024

(30) Foreign Application Priority Data
Nov. 11, 2021 (KR) 10-2021-0155208

(51) Int. Cl. C12N 9/52

(52) U.S. Cl. CPC C12N 9/52; C12Y 304/24069

(57) ABSTRACT

The present invention relates to a method for drying botulinum toxin under reduced pressure. In the case of using a method for preparing a botulinum toxin dried cake according to this method for drying under reduced pressure, the drying time can be significantly shortened while maintaining the efficacy of the botulinum toxin.

### TECHNICAL FIELD

The present invention relates to a drying process for a toxin formulation, and specifically, to a method for preparing a botulinum toxin dried cake by a reduced pressure drying method.

### BACKGROUND ART

Botulinum toxin (BTX) is a neurotoxin produced by an anaerobic bacterium called Clostridium botulinum (C. botulinum). There exists a total of 7 types of botulinum toxin (types A to G), of which two types, i.e., botulinum type A (BTX-A) and type B (BTX-B), have been purified and are being medically used.

Botulinum toxin blocks muscle contraction signal transduction to relax muscles by inhibiting the release of acetylcholine, a neurotransmitter. In other words, it blocks the release of acetylcholine, a neurotransmitter secreted from the presynaptic terminal of the neuromuscular junction, causing nerve paralysis. While fillers are medical materials that fill an area with insufficient skin volume, botulinum toxin preparation differs from fillers in that it is a medication containing ingredients that deteriorate activity of muscle by blocking the release of neurotransmitters that cause muscle contraction.

Botulinum toxin is mainly used to suppress or remove wrinkles between the eyebrows and around the eyes, as well as to treat upper limb stiffness of stroke, eyelid spasms, equinus deformity, etc. Botulinum toxin is gradually expanding its scope of indications.

Commercially available botulinum toxin products may be provided in solution form with excipients containing sodium chloride and human serum albumin or in solid form (i.e., dried cakes) after a drying process. Most botulinum toxin manufacturers produce products that have undergone a freeze-drying process. For example, there are Meditoxin®, Xeomin®, Dysport®, etc.

Meanwhile, in the drying process of botulinum toxin, many conventional freeze drying methods have been known (such as KR 10-2012-0112248 A, etc.), but the methods take quite a long time (approximately 18 to 48 hours) since they essentially involve the freezing process and moisture is removed through the sublimation process. In particular, the freezing process, which is an essential process of the freeze-drying process, may cause a decrease in activity through damage to the protein structure due to ice nuclei formation or an imbalance of excipient concentration that occurs partially during freezing.

This freeze-drying process is generally known as an advanced type of drying method for dry formulations of protein pharmaceuticals. However, it is unsuitable for botulinum toxin formulations in which extremely small amounts of protein are used.

Therefore, there is a need to develop a drying process suitable for the characteristics of botulinum toxin formulations. In addition, a method is needed to efficiently use the long process time for primary and secondary drying due to the characteristics of the freeze-drying process.

### DETAILED DESCRIPTION

### Technical Objective

The inventors conducted an intensive research to develop an optimized, reduced pressure drying method for botulinum toxin that maintains effectiveness and stability. As a result, the inventors successfully demonstrated that by efficiently controlling parameters related to the drying process, such as pressure and (shelf) temperature, the protein botulinum toxin can be protected from external stimuli occurring during the process and the drying time can be significantly shortened.

Therefore, the purpose of the present invention is to provide a method for preparing a botulinum toxin dried cake.

### Means for Solving the Problems

The inventors conducted an intensive research to develop an optimized, reduced pressure drying method for botulinum toxin that maintains effectiveness and stability. As a result, the inventors successfully demonstrated that by efficiently controlling parameters related to the drying process, such as pressure and (shelf) temperature, the protein botulinum toxin can be protected from external stimuli occurring during the process and the drying time can be significantly shortened.

The present invention relates to a method for preparing a botulinum toxin dried cake.

Hereinafter, the present invention will be discussed in more detail.

According to one aspect of the present invention, the present invention provides a method for preparing a botulinum toxin dried cake, comprising the step of drying botulinum toxin under reduced pressure at a pressure of 1,500 to 60,000 mTorr and a temperature of 3° C. to 25° C.

As used herein, the term “botulinum toxin (BTX)” refers to a toxin that can be obtained by purifying it from a Clostridium botulinum (C. botulinum) strain. The botulinum toxin produced by Clostridium botulinum (C. botulinum) strains has a size of about 150 kDa. A botulinum toxin complex, which is a complex of botulinum toxin and one or more non-toxin proteins, has a size of about 300 kDa to 900 kDa.

The above botulinum toxin may be used for various medical purposes, as well as suppressing or improving wrinkles between the eyebrows and those around the eyes.

As used herein, the term “cake” refers to a dried material on the bottom surface of a vial in dry formulation, which may be used separately from a powder formulation.

In one embodiment of the present invention, a strain of the present invention producing botulinum toxin is Clostridium botulinum (C. botulinum). More specifically, the Clostridium botulinum Type A (ATCC 19397) strain may be used, but the strain of the present invention is not limited thereto. Botulinum toxin type A has been approved by the U.S. Food and Drug Administration (FDA) for the treatment of essential blepharospasm, strabismus, and hemifacial spasm in patients over 12 years of age. It also has been approved for the treatment of cervical dystonia, glabellar (facial) wrinkles, and hyperhidrosis.

In another embodiment of the present invention, various conventionally known processes may be used to produce the botulinum toxin of the present invention, and the process for the present invention is not particularly limited.

Specifically, for example, it may involve a strain culture medium producing botulinum toxin that has been cultured, and subjected to precipitation, filtering, re-dissolution, and purification processes once or at least two times.

More specifically, for example, the botulinum toxin of the present invention may be a strain culture medium producing botulinum toxin prepared by purifying it into a crystalline complex which consists of an active high molecular weight toxin protein and a relevant erythrocyte agglomerate protein, through a series of acid precipitations of a botulinum toxin complex from a culture of Clostridium botulinum (C. botulinum) cultured in a specific medium obviously understood by those skilled in the art as suitable for culturing Clostridium botulinum (C. botulinum), and dissolving the purified crystalline complex in a solution comprising brine and a stabilizer.

The culture conditions of the strain culture medium producing botulinum toxin may be appropriately adjusted based on the general knowledge of a person skilled in the relevant art depending on the culture environment.

In another embodiment of the present invention, the concentration of the botulinum toxin of the present invention may be 250 U/mL to 5,000 U/mL.

Specifically, in a preferred embodiment of the present invention, the concentration of the botulinum toxin may be 250 U/mL to 5,000 U/mL, 300 U/mL to 5,000 U/mL, 400 U/mL to 5,000 U/mL, 500 U/mL to 5,000 U/mL, 600 U/mL to 5,000 U/mL, 700 U/mL to 5,000 U/mL, 800 U/mL to 5,000 U/mL, 900 U/mL to 5,000 U/mL, 1,000 U/mL to 5,000 U/mL, 1,000 U/mL to 4,500 U/mL, 1,000 U/mL to 4,000 U/mL, 1,000 U/mL to 3,500 U/mL, 1,000 U/mL to 3,000 U/mL, 1,000 U/mL to 2,500 U/mL, 1,000 U/mL to 2,000 U/mL, 1,000 U/mL to 1,500 U/mL, 1,000 U/mL to 1,200 U/mL, 800 U/mL to 1,200 U/mL, 800 U/mL to 1,100 U/mL, 800 U/mL to 1,000 U/mL, 800 U/mL to 900 U/mL, 900 U/mL to 1,200 U/mL, 1,000 U/mL to 1,200 U/mL, or 1,100 U/mL to 1,200 U/mL.

In another embodiment of the present invention, the method for preparing the botulinum toxin dried cake of the present invention may involve drying the above-described botulinum toxin under reduced pressure at a pressure of 1,500 or more and less than 60,000 mTorr.

Specifically, in a preferred embodiment of the present invention, the drying under reduced pressure of the present invention may be performed at a pressure of 1,500 to 60,000 mTorr, 1,500 to 55,000 mTorr, 1,500 to 50,000 mTorr, 1,500 to 45,000 mTorr, 1,500 to 40,000 mTorr, 1,500 to 35,000 mTorr, 1,500 to 30,000 mTorr, 1,500 to 25,000 mTorr, 1,500 to 20,000 mTorr, 1,500 to 15,000 mTorr, 1,500 to 14,000 mTorr, 1,500 to 13,000 mTorr, 1,500 to 12,000 mTorr, 1,500 to 11,000 mTorr, 1,500 to 10,000 mTorr, 1,500 to 9,000 mTorr, 1,500 to 8,000 mTorr, 1,500 to 7,000 mTorr, 1,500 to 6,000 mTorr, 1,500 to 5,000 mTorr, 1,500 to 4,000 mTorr, 1,500 to 3,500 mTorr, 1,500 to 3,000 mTorr, 1,500 to 2,500 mTorr, 1,500 to 2,000 mTorr, 2,500 to 3,000 mTorr, 2,500 to 3,500 mTorr, 2,500 to 4,000 mTorr, 2,500 to 4,500 mTorr, 2,500 to 5,000 mTorr, 2,500 to 6,000 mTorr, 2,500 to 7,000 mTorr, 2,500 to 8,000 mTorr, 2,500 to 9,000 mTorr, 2,500 to 10,000 mTorr, 2,500 to 11,000 mTorr, 2,500 to 12,000 mTorr, 2,500 to 13,000 mTorr, 2,500 to 14,000 mTorr, 2,500 to 15,000 mTorr, 2,500 to 20,000 mTorr, 2,500 to 25,000 mTorr, 2,500 to 30,000 mTorr, 2,500 to 35,000 mTorr, 2,500 to 40,000 mTorr, 2,500 to 45,000 mTorr, 2,500 to 50,000 mTorr, 2,500 to 55,000 mTorr, or 2,500 to 60,000 mTorr.

If it deviates from the above ranges, protein damage may occur due to phenomena such as boiling over, and a perfectly dried product may not be obtained because sufficient drying is not feasible.

Meanwhile, in the above drying under reduced pressure, it is necessary to reach the target pressure at a constant speed in a stably controlled state from normal [atmospheric] pressure, which is general atmospheric conditions.

In another embodiment of the present invention, the method for preparing the botulinum toxin dried cake of the present invention may involve drying the above-described botulinum toxin under reduced pressure at a temperature of 3 to 25° C.

Specifically, in a preferred embodiment of the present invention, the drying under reduced pressure of the present invention may be performed at a temperature of 3° C. to 25° C., 5° C. to 25° C., 7° C. to 25° C., 9° C. to 25° C., 11° C. to 25° C., 12° C. to 25° C., 3° C. to 20° C., 5° C. to 20° C., 7° C. to 20° C., 9° C. to 20° C., 11° C. to 20° C., 12° C. to 20° C., 3° C. to 18° C., 3° C. to 16° C., 3° C. to 14° C., or 3° C. to 12° C.

If it deviates from the above ranges, due to higher or lower temperatures, the structural instability of the botulinum toxin may increase or physical damage and deformation thereto may occur, which may lead to a decrease in efficacy.

In another embodiment of the present invention, the method for preparing the botulinum toxin dried cake of the present invention may be performed until the humidity of the botulinum toxin dried cake of the present invention reaches 3% or less.

Specifically, the method for preparing the botulinum toxin dried cake of the present invention may involve drying the above-described botulinum toxin under reduced pressure for 0.5 hours or more, preferably 0.5 to 4 hours.

Specifically, in a preferred embodiment of the present invention, the drying under reduced pressure of the present invention may be performed in the range of 0.5 hours to 4 hours, 0.5 hours to 3 hours, 0.5 hours to 2 hours, 0.5 hours to 1 hour, 1 hour to 4 hours, 2 hours to 4 hours, or 3 hours to 4 hours.

The botulinum toxin dried cake, i.e., the final product prepared by the method for preparing a botulinum toxin dried cake of the present invention, may have a standard titer of 80% to 120, or 85% to 115%.

In the process of manufacturing the botulinum toxin dried cake of the present invention, depending on volume or surface area of a solvent for drying the product, boiling over may occur due to the amount of dissolved oxygen or change in concentration of the composition concentrated during the drying process, and unexpected ice crystal formation may occur due to changes in freezing point. If the product is dried under these conditions, the product quality may be deteriorated due to the irregularity in the product's properties and protein damages. Therefore, it is preferable for the drying process to be performed under the conditions that volume or surface area of a solvent for drying the product is appropriately adjusted, which can be easily controlled and performed by a person skilled in the art who understood the present specification.

The method for preparing a botulinum toxin dried cake of the present invention utilizes the above-described drying process under reduced pressure, by which the conventional freeze-drying process can be disused. In one aspect of the present invention, the present invention uses a drying process under reduced pressure, which shortens the processing time and minimizes damages to the protein drug product during drying. Accordingly, the present invention has the advantages in that it is more efficient and commercially more feasible compared to the conventional technologies.

### Working Effect of the Invention

The present invention relates to a method for drying botulinum toxin under reduced pressure. When the method for preparing a botulinum toxin dried cake of the present invention by drying under reduced pressure is used, the drying time can be significantly shortened while the efficacy of the botulinum toxin is maintained.

### BRIEF DESCRIPTION OF DRAWINGS

FIG. 1 demonstrates the difference in cake properties between reduced pressure-dried product and freeze-dried product.

FIGS. 2a and 2b demonstrate the possibility of denaturation and loss of botulinum toxin according to conventional low vacuum, reduced pressure drying conditions.

FIGS. 3a and 3b demonstrate the difference in cake properties (FIG. 3a) and the change in temperature within the vial (FIG. 3b) according to pressure conditions in the range of 1,000 to 70,000 mTorr in the drying under reduced pressure of the present invention.

FIG. 4 demonstrates the change in temperature within the vial according to pressure conditions in the range of 50,000 to 70,000 mTorr in the drying under reduced pressure of the present invention.

### MODES FOR CARRYING OUT THE INVENTION

Hereinafter, the present invention will be described in more detail based on the working examples. The working examples are presented merely in order to elaborate the present inventive concept more specifically, and it should be obvious to a person skilled in the art that in view of the gist of the present invention, the scope of the present invention should not be construed as being limited to the working examples as presented.

Unless otherwise indicated in the examples, “toxin” or “botulinum toxin” refers to the botulinum toxin type A complex with a molecular weight of about 900 kDa. The method disclosed herein has the applicability prepared for formulations of toxins, complexes, botulinum toxin serotypes, and botulinum neurotoxin components of about 150 kDa, about 300 kDa, and about 500 kDa as well as other molecular weights.

### Preparation Example: Preparation of a Final Stock Solution of Botulinum Toxin

The final stock solution of botulinum toxin was prepared by adding 0.9% sodium chloride (Merck, 1.37017.5000), 0.5% human serum albumin (Green Cross, 161B19508), and 1000 U/mL botulinum toxin type A stock solution (Inibio Co., Ltd., Korea).

### Comparative Example 1: Comparison of Titer of Botulinum Toxin with Freeze-Drying

### 1-1. Drying Experiment

Formulations dried under freeze-drying conditions and those dried under reduced pressure drying conditions were compared, respectively.

Freeze-drying conditions and reduced pressure drying conditions are indicated in Table 1 below.

TABLE 1

| | | Shelf | | |
|---|---|---|---|---|
| | Process | temperature | Time | Pressure |
| Drying method | Name | (° C.) | (m) | (mTorr) |
| Freeze-drying | Freezing | −50 | 180 | |
| | 1st Drying | −50 | 480 | 200 |
| | 2nd Drying | 20 | 180 | 100 |
| Reduced pressure | Drying | 11.5 | 60 | 2500 |
| drying | | | | |

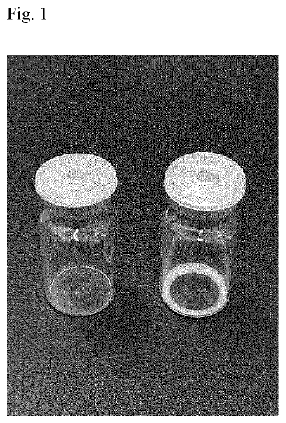

As shown in FIG. 1, in the case of freeze drying (right vial in FIG. 1), a thicker white dried cake was formed compared to reduced pressure drying (left vial in FIG. 1). This dried cake was easily breakable due to the impact applied to the vial, and the shattering of the broken dried cake could occur.

### 1-2. Titer Experiment

Regarding the final stock solution of botulinum toxin prepared in the above preparation example, the formulation dried under freeze drying conditions (see Table 2 below) was dissolved in 2.8 mL of physiological saline and administered to the abdominal cavity (0.1 mL/mouse) of ten ICR-mice (4 weeks old, body weight 18-22 g; Coretech Co., Ltd., Korea) respectively. The numbers of dead animals and living animals were checked for 3 days. Titers were calculated using a statistical program (CombiStats 6.1, EDQM). The appropriate titer standard was set to a range that includes the error range of animal testing (see Table 3 below). The results are shown in Table 4 below.

TABLE 2

| | Shelf temperature | Time | Pressure |
|---|---|---|---|
| Process Name | (° C.) | (m) | (mTorr) |
| Freezing | −50 | 180 | |
| 1st Drying | −50 | 480 | 200 |
| 2nd Drying | 20 | 180 | 100 |

TABLE 3

| Experiment | Standard Titer Range(%) |
|---|---|
| Titer experiment | 100 ± 15% |

TABLE 4

| Vial No. | Drying condition | Standard Titer (%) |
|---|---|---|
| # 1 (Comparative Example 1) | Freeze drying | 65.0 |
| # 2 (Comparative Example 2) | Freeze drying | 56.3 |
| # 3 (Comparative Example 3) | Freeze drying | 56.5 |

As shown in Table 4, the titer of the botulinum toxin formulations to which a conventional freeze drying process was performed was 56-65%, which is outside the range shown in Table 3 above, indicating that the efficacy was reduced due to protein damages.

The above results indicate that the formulation of botulinum toxin dried under reduced pressure can retain superior activity of botulinum toxin compared to the freeze-dried formulation.

### Reference Example: Confirmation of Denaturation and Loss of Botulinum Toxin Due to Reduced Pressure Drying

The final stock solution of botulinum toxin prepared in the above preparation example was filled into vials by 5 mL or 1 mL, and the physical changes and freezing point changes depending on changes in conventional low-vacuum drying pressure were examined.

Specifically, the reduced pressure drying was performed by changing the pressure from 1 bar to 0.0019 bar (about 1500 mTorr). The shelf temperature was set to 5° C. Distilled water (DW) and 0.9% NaCl solution were used as control groups.

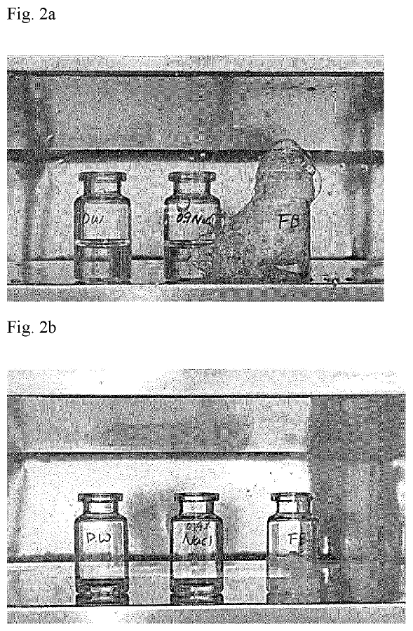

FIG. 2a shows the phase changes by pressure after 5 mL of each solution was filled into a vial. Under the above conditions, the boiling phenomenon of the solution occurred in the final stock solution of botulinum toxin below 10,000 mTorr, but it did not occur in the control group.

FIG. 2b shows the phase changes by pressure after 1 mL of each solution was filled into a vial. Under the above conditions, the boiling over and ice crystal formation of the final stock solution occurred at a pressure of 1,500 mTorr, but they did not occur in the control group.

This phenomenon is understood to have been occurred because volume or surface area of the solvent was not suitable for the characteristics and drying conditions of the solvent. This may make it difficult to properly control the drying process or cause sublimation rather than complete evaporation due to ice crystals generated during the process, making it difficult to perform a normal reduced pressure drying. In addition, if drying of the product proceeds under uncontrollable conditions as above, the product quality may be deteriorated due to non-uniformity of product properties and damages to proteins.

### Example 1: Establishment of Reduced Pressure Drying Conditions Based on the Titer of Botulinum Toxin

For the formulation in which 0.1 mL of the final stock solution of botulinum toxin prepared in the above preparation example was filled into vials and reduced pressure drying was conducted under the conditions shown in Table 5 below, the titer was calculated in the same manner as in Comparative Example 1-2. The appropriate titer standard was set to a range that includes the error range of animal testing (see Table 3 below), and the results are shown in Table 5 below.

TABLE 5

| | Pressure | Shelf Temperature | Standard Titer |
|---|---|---|---|
| Vial No. | (mTorr) | (° C.) | (%) |
| # 4 | 1500 | 3 | 99.5 |
| # 5 | 1500 | 3 | 97.7 |
| # 6 | 2500 | 3 | 108.8 |
| # 7 | 3500 | 3 | 113.5 |
| # 8 | 3500 | 5 | 102.8 |
| # 9 | 1500 | 12 | 84.4 |
| # 10 | 2500 | 11.5 | 94.8 |
| # 11 | 3500 | 11.5 | 105.8 |
| # 12 | 1500 | 20 | 91.6 |
| # 13 | 2500 | 20 | 95.1 |
| # 14 | 3500 | 20 | 100.4 |
| # 15 | 3500 | 20 | 105.9 |
| # 16 (Comparative | 500 | 25 | 81.6 |
| Example 4) | | | |
| # 17 (Comparative | 70000 | 25 | 91.0 |
| Example 5) | | | |

As shown in Table 5, the titer of the botulinum toxin formulation that was dried for 1 hour at 1,500 to 30,000 mTorr and 3 to 25° C. satisfied the range shown in Table 3. On the other hand, when the drying process was performed in a range outside 1,500 to 30,000 mTorr (Comparative Examples 4 and 5), it was found that the titer of the botulinum toxin formulation did not satisfy the appropriate range.

### Example 2: Drying Time Under the Reduced Pressure Drying Conditions of the Present Invention #1

Generally, drying can be performed in two steps: the separation of water molecules connected by hydrogen bonds, and the removal of water molecules variously interacting with other excipients and proteins that make up the final stock solution. When water molecules form bonds with each other, they can be separated with relatively less energy than water molecules interacting with other molecules. In most cases, water molecules are bonded to each other, and primary evaporation is performed by separating them. In this process, the liquid product shows a process of changing into a gaseous phase. Secondary evaporation occurs when the bonds in the interaction between water molecules and other excipients are broken, and it is determined that drying is completed if this process is almost completed. As a result of humidity measurement, the humidity must be within 3% to meet the standards for dried cake injections.

Therefore, the speed of moisture evaporation or suitability can be confirmed through the phase change of the product.

### 2-1. Time for Phase Change (Liquid→Gas) at Each Pressure Condition

Based on the results of Example 1, the empty vials were filled with the final stock solution of the botulinum toxin prepared in the above preparation example by 0.1 mL each, and time for the phase change at each pressure condition was examined.

Specifically, as shown in Table 6 below, the drying process was performed for 1 hour at conditions of 1,000 to 70,000 mTorr and 25° C. Change in temperature due to the phase change was measured by a temperature sensor installed in the vial. The speed at which the phase change occurs was faster with lower pressure, by which it was confirmed that evaporation was occurring.

TABLE 6

| | Pressure | Time for phase change |
|---|---|---|
| Vial No. | (mTorr) | (min) |
| # 18 (Comparative | 1,000 | 11 |
| Example 6) | | |
| # 19 | 3,000 | 13 |
| # 20 | 10,000 | 15 |
| # 21 | 30,000 | 43 |
| # 22 | 50,000 | — |
| # 23 | 60,000 | — |
| # 24 (Comparative | 70,000 | — |
| Example 7) | | |

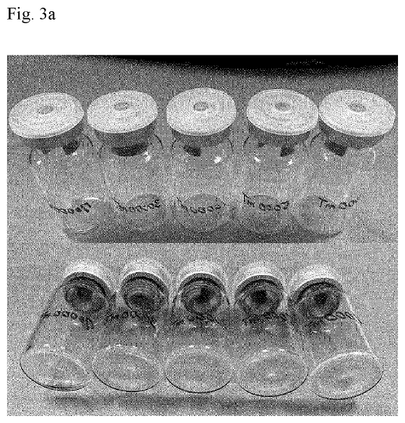

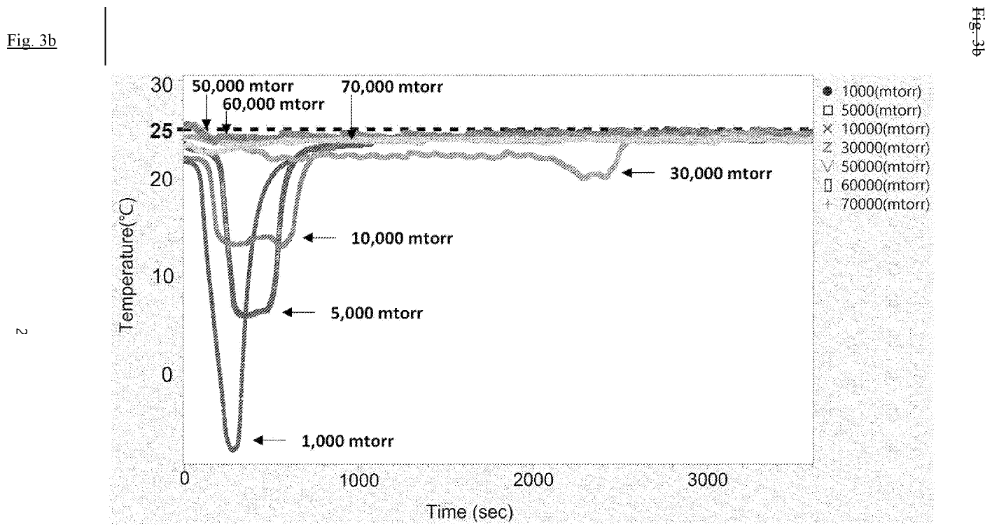

As shown in Table 6 and FIGS. 3a and 3b, a white dried cake was formed on the bottom of most vials, which results from the phase change due to evaporation. The time taken for the phase change to occur was found to be 11 to 43 minutes. However, at 50,000 mTorr, 60,000 mTorr and 70,000 mTorr, this phenomenon was not observed within 1 hour.

The above results suggest that at higher pressures, the time required for complete drying (evaporation) will increase, and that preparation of the botulinum toxin dried cake pursued by the present invention may be difficult at pressures exceeding a certain range (Comparative Example 7).

### 2-2. Humidity of the Botulinum Toxin Dried Cake as Produced

Based on the results of Example 1 and 2-1, the empty vials were filled with the final stock solution of the botulinum toxin prepared in the above preparation example by 0.1 mL each. For the vials containing the botulinum toxin dried cake that was dried under reduced pressure at 3,500 mTorr and 5° C., the moisture was measured in an environment with a humidity of 10% or less. The humidity (%) was calculated using the following calculation formula, and the appropriate humidity standards are shown in Table 7 below with reference to the Pharmacopoeia.

$$Humidity ( \% ) = \frac{\begin{matrix}Moisture content of botulinum toxin dried cake - \\ Moisture content of empty vial\end{matrix}}{Weight of botulinum toxin dried cake} \times 100 \% \qquad [ Formula ]$$

TABLE 7

| Experiment | Range | Reference |
|---|---|---|
| Humidity experiment | 3% or less | USP, EP |

TABLE 8

| Vial No. | Time (h) | Humidity (%) |
|---|---|---|
| # 25 | 0.5 | 1.01 |
| # 26 | 1 | 1.53 |
| # 27 | 4 | 1.49 |

As shown in Table 8, the humidity of the botulinum toxin formulation after the drying process for 0.5 to 4 hours satisfied the range shown in Table 7 above.

### Example 3. Drying Time Under the Reduced Pressure Drying Conditions of the Present Invention #2

Based on the results of Example 2, the empty vials were filled with the final stock solution of the botulinum toxin prepared in the above preparation example by 0.1 mL each, and time for the phase change at each pressure condition was examined.

Specifically, as shown in Table 9 below, the drying process was performed for 3 hours at conditions of 50,000 to 70,000 mTorr and 25° C. Change in temperature due to the phase change was measured by a temperature sensor installed in the vial. The speed at which the phase change occurs was faster with lower pressure, by which it was confirmed that evaporation was occurring.

TABLE 9

| Vial No. | Pressure (mTorr) | Time for phase change (min) |
|---|---|---|
| # 28 | 50,000 | 143 |
| # 29 | 60,000 | 183 |
| # 30 (Comparative | 70,000 | — |
| Example 8) | | |

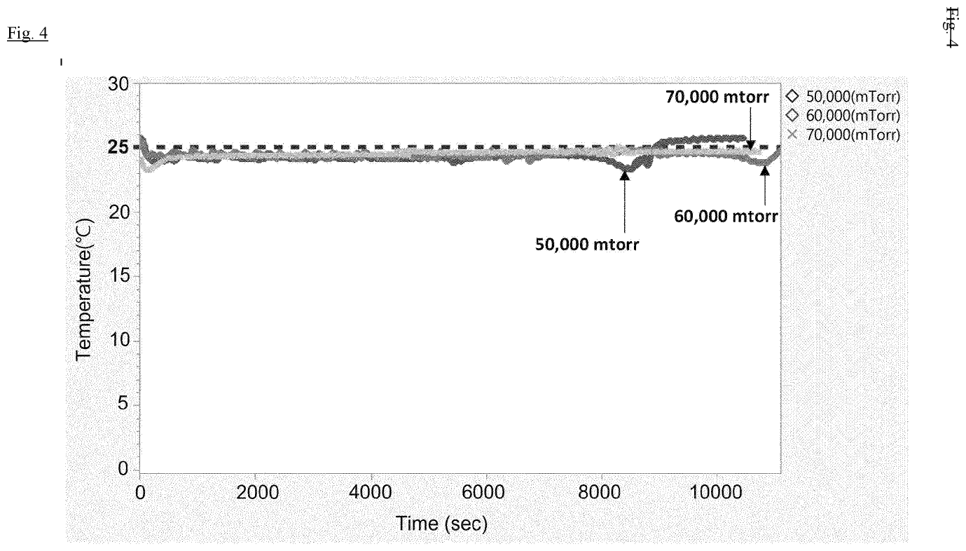

As shown in Table 9 and FIG. 4, the time taken for the phase change to occur under 50,000 and 60,000 mTorr conditions was 143 minutes and 183 minutes, respectively. However, at 70,000 mTorr, this phenomenon was not observed.

The above results suggest that at higher pressures, the time required for complete drying (evaporation) will increase, and that preparation of the botulinum toxin dried cake pursued by the present invention may be difficult at pressures exceeding a certain range (Comparative Example 8).

1. A method for preparing a botulinum toxin dried cake, comprising the step of drying the botulinum toxin under reduced pressure at pressure conditions of 1,500 mTorr to 60,000 mTorr.

2. The method of claim 1, characterized in that the drying under reduced pressure is performed at temperature conditions of 3° C. to 25° C.

3. The method of claim 1, characterized in that the drying under reduced pressure is performed for 0.5 hours or more, or 0.5 to 4 hours.

4. The method according to claim 1, characterized in that concentration of the botulinum toxin is 250 U/mL to 5,000 U/mL.

5. The method according to claim 1, characterized in that the produced botulinum toxin dried cake has a standard titer of 80% to 120%, or 85% to 115%.

6. The method according to claim 1, characterized in that drying under reduced pressure is performed until humidity of the botulinum toxin dried cake reaches 3% or less.

7. The method according to claim 1, characterized in that a strain producing botulinum toxin is Clostridium botulinum (C. botulinum) type A.

8. The method according to claim 1, characterized in that the pressure conditions for drying under reduced pressure are determined by reaching a target pressure at a constant speed in a stably controlled state from normal pressure.

9. The method according to claim 1, characterized in that the drying under reduced pressure is performed at a pressure of 1,500 to 60,000 mTorr, 1,500 to 55,000 mTorr, 1,500 to 50,000 mTorr, 1,500 to 45,000 mTorr, 1,500 to 40,000 mTorr, 1,500 to 35,000 mTorr, 1,500 to 30,000 mTorr, 1,500 to 25,000 mTorr, 1,500 to 20,000 mTorr, 1,500 to 15,000 mTorr, 1,500 to 14,000 mTorr, 1,500 to 13,000 mTorr, 1,500 to 12,000 mTorr, 1,500 to 11,000 mTorr, 1,500 to 10,000 mTorr, 1,500 to 9,000 mTorr, 1,500 to 8,000 mTorr, 1,500 to 7,000 mTorr, 1,500 to 6,000 mTorr, 1,500 to 5,000 mTorr, 1,500 to 4,000 mTorr, 1,500 to 3,500 mTorr, 1,500 to 3,000 mTorr, 1,500 to 2,500 mTorr, 1,500 to 2,000 mTorr, 2,500 to 3,000 mTorr, 2,500 to 3,500 mTorr, 2,500 to 4,000 mTorr, 2,500 to 4,500 mTorr, 2,500 to 5,000 mTorr, 2,500 to 6,000 mTorr, 2,500 to 7,000 mTorr, 2,500 to 8,000 mTorr, 2,500 to 9,000 mTorr, 2,500 to 10,000 mTorr, 2,500 to 11,000 mTorr, 2,500 to 12,000 mTorr, 2,500 to 13,000 mTorr, 2,500 to 14,000 mTorr, 2,500 to 15,000 mTorr, 2,500 to 20,000 mTorr, 2,500 to 25,000 mTorr, 2,500 to 30,000 mTorr, 2,500 to 35,000 mTorr, 2,500 to 40,000 mTorr, 2,500 to 45,000 mTorr, 2,500 to 50,000 mTorr, 2,500 to 55,000 mTorr, or 2,500 to 60,000 mTorr.

10. The method according to claim 1, characterized in that the drying under reduced pressure is performed at a temperature of 5° C. to 25° C., 7° C. to 25° C., 9° C. to 25° C., 11° C. to 25° C., 12° C. to 25° C., 3° C. to 20° C., 5° C. to 20° C., 7° C. to 20° C., 9° C. to 20° C., 11° C. to 20° C., 12° C. to 20° C., 3° C. to 18° C., 3° C. to 16° C., 3° C. to 14° C., or 3° C. to 12° C.

11. The method according to claim 1, characterized in that the drying under reduced pressure is performed in a range of 0.5 hours to 4 hours, 0.5 hours to 3 hours, 0.5 hours to 2 hours, 0.5 hours to 1 hours, 1 hours to 4 hours, 2 hours to 4 hours, or 3 hours to 4 hours.
